(19) United States
(12) Patent Application Publication
(10) Pub. No.: US 2007/0087050 A1
(43) Pub. Date: Apr. 19, 2007

(54) Orally Disintegratable Simvastatin Tablets

(76) Inventor: Korinde Annemarie Jansen, Loo (NL)

Correspondence Address: SYNTHON IP INC, 7130 HERITAGE VILLAGE PLAZA, STE 202, GAINESVILLE, VA, 20155, US

(21) Appl. No.: 11/465,418

(22) Filed: Aug. 17, 2006

Related U.S. Application Data
(60) Provisional application No. 60/708,773, filed on Aug. 17, 2005.

(51) Int. Cl. A61K 31/22; A61K 9/20

(52) U.S. Cl. 424/464; 514/548

(57) ABSTRACT

An orally disintegratable tablet containing simvastatin and silicified microcrystalline cellulose is provided with a non-alkaline lubricant.

This application claims the benefit of priority under 35 U.S.C. § 119(e) from U.S. provisional application serial No. 60/708,773, filed Aug. 17, 2005, the entire contents of which are incorporated herein by reference.

### BACKGROUND OF THE INVENTION

The present invention relates to orally disintegrating tablets that contain simvastatin as the active ingredient and silicified microcrystalline cellulose as a matrix-forming agent.

Orally disintegrating dosage forms for delivery of pharmaceuticals are known in the art. The purpose of such systems is to allow administration of a solid dosage form, for instance a tablet, of a beneficial drug to a patient without the need to swallow the dosage form. The orally disintegrating tablet should disintegrate and, optionally dissolve, directly in the oral cavity, with the aid of saliva or, in some cases a small amount of water. The resulting liquid or dispersion is then easily swallowed. This causes easy and immediate entry of the dissolved or dispersed beneficial drug into the gastrointestinal tract. In some cases the drug may even be absorbed by the oral mucosa or the esophageal lining as it passes down to the stomach. Orally disintegrating tablets, contrary to candies or sublingual tablets, should disintegrate in a time not exceeding one minute or so in the oral cavity. In general, there are many types of patients that could benefit from orally disintegrating dosage forms, such as pediatric patients, geriatric patients, patients with renal disorders, patients with swallowing disorders, etc. Indeed, dysphagia or difficulty in swallowing is seen to afflict nearly 35% of the general population.

An orally disintegratable tablet composition was disclosed in commonly owned U.S. Patent Application Publication No. US-2004-0265375-A1, the entire contents of which are incorporated herein by reference. The Publication teaches that silicified microcrystalline cellulose can be used to form orally disintegrating tablets. The tablet composition comprises a pharmaceutical active agent, at least 50% of a silicified microcrystalline cellulose, and optionally other excipients including a lubricant. The Publication specifically mentions sodium stearyl fumarate and magnesium stearate as useful lubricants, the former being preferred as tending to facilitate faster dissolution rates. Among the pharmaceutically active agents mentioned as being suitable for use in the orally disintegrating tablet is simvastatin, a known antihypercholesterolemic compound. Simvastatin was described in U.S. Pat. No. 4,444,784 and is sold commercially in a conventional tablet dosage from by MERCK. Further, Example 16 of the US-2004-0265375-A1 Publication exemplifies a simvastatin orally disintegratable tablet. The example 16 composition is shown below.

| | mg/tablet |
|---|---|
| Simvastatin | 10.00 |
| BHA (butylated hydroxyanisol) | 0.02 |
| Sodium starch glycolate | 0.34 |
| Povidon (PVP) | 0.66 |
| Silicified microcrystalline cellulose | 49.46 |
| L-HPC | 4.20 |
| aspartame | 2.10 |
| mint flavor | 2.10 |
| Sodium stearyl fumarate | 1.05 |
| Iron oxide yellow | 0.07 |
| Tablet weight | 70 |

The tablets were taught to be made by granulating the simvastatin, BHA, sodium starch glycolate and povidon in a high shear granulator. The granulate was sieved and dried. The dried granulate was mixed with the silicified microcrystalline cellulose, L-HPC, aspartame, mint flavor and iron oxide yellow in a free-fall mixer. After addition of the sodium stearyl fumarate (the lubricant) the mixing was finalized. Oval biconvex tablets with a diameter of 7 mm were prepared on an EK0 tablet press. The disintegration time of the tablets as measured by the Ph. Eur. disintegration test was less than 30 seconds. Although the orally disintegratable tablets described in the 0265375 Publication are successful in orally disintegrating and for administering the pharmaceutical active agent, it has been subsequently discovered that simvastatin-containing tablets based on the above composition would benefit from improved stability during storage. Thus, there is a need to find a more stable orally disintegratable tablet formulation of simvastatin.

### SUMMARY OF THE INVENTION

The present invention involves the discovery that orally disintegratable pharmaceutical tablets comprising simvastatin and silicified microcrystalline cellulose can have improved stability by using a non-alkali lubricant. Accordingly, a first aspect of the invention relates to an orally disintegratable pharmaceutical tablet comprising simvastatin, at least 50 wt % of a silicified microcrystalline cellulose, and a non-alkali lubricant.

Another aspect of the invention relates to an orally disintegratable pharmaceutical tablet comprising simvastatin, at least 50 wt % of silicified microcrystalline cellulose, and a lubricant, the improvement of which comprises that said composition does not contain sodium stearyl fumarate and preferably does not contain an alkali lubricant.

A further aspect of the invention relates to a method of treating high cholesterol by administering a tablet as described above. This can be accomplished in a variety of ways such as orally administering the orally disintegratable pharmaceutical tablet to a patient in need thereof. Alternatively, the process can comprise disintegrating a tablet as described above in a liquid to a form a simvastatin-containing liquid and orally administering the simvastatin-containing liquid to a patient in need thereof.

### DESCRIPTION OF THE INVENTION

The present invention relates to the discovery of a stability issue associated with the use of simvastatin in a silicified microcrystalline cellulose-based orally disintegratable tablet and to the discovery of the solution thereto. More specifically, replacing the previously preferred lubricant of the US-2004-0265375-A1 Publication, namely sodium stearyl fumarate, with another lubricant was found to improve the stability of the formulation. It is unclear why the sodium stearyl fumarate aides the degradation pathway of simvastatin; e.g., the high pH of sodium stearyl fumarate, a unique reaction site, etc. Nonetheless, removing this relatively alkaline lubricant reduced the degradation. The replacement lubricants are generally non-alkali lubricants, which are defined hereinafter.

As used herein the expression “orally disintegratable” means that the tablet disintegrates or disperses within less than 90 seconds as measured by the in vitro disintegration test described in US Pharmacopoeia 701, without disks. Such a disintegration test result is reasonably related to the actual disintegration time experienced by a mammal when placed in the oral cavity (albeit placement within such a cavity is not required). Preferably, the tablets of the present invention disintegrate in less than 80 seconds, more preferably less than 60 seconds including less than 50 seconds and even less than 40 seconds, and most preferably in less than 30 seconds. In some preferred embodiments, the disintegration occurs within the range of 1 to 30 seconds, more preferably 1 to 20 seconds, still more preferably 1 to 15 seconds, and frequently within 1 to 10 seconds. It should be noted that the corresponding European Pharmacopoeia method generally provides similar results to the above-quoted USP method.

The orally disintegratable tablets of the present invention contain simvastatin. The amount of the simvastatin in a single tablet is generally effective for its intended purpose. The effective amount is typically within the range of 2 to 200 mg, more particularly 5, 10, 20, 40 or 80 mg, per tablet. Simvastatin is a commercially available drug substance and can also be made by various techniques known in the art. Although the particle size of the simvastatin is not particularly limited in the present invention, it is generally desirable to use a smaller particle size than is typically used in making conventional tablet dosage forms. Specifically, the smaller particle size can enhance the bioavailability to the desired level. For the orally disintegratable tablets of the present invention, it is preferred that all of the simvastatin particles have a size of 10 microns or less, more typically 8 microns or less and/or that 90% of the simvastatin particles have a size of 8 microns or less, more typically 5 microns or less; i.e. d₉₀≦5 microns. Simvastatin particles having these kinds of reduced sizes, e.g., micronized simvastatin, could be obtained by controlling the precipitation conditions, but more typically are obtained by milling and/or sieving simvastatin particles.

The orally disintegratable tablets of the invention also contain silicified microcrystalline cellulose (referred to sometimes hereinafter as “silicified cellulose”). Silicified microcrystalline cellulose is an intimate physical mixture of colloidal silicon dioxide with microcrystalline cellulose as described in U.S. Pat. No. 5,585,115. It is not merely an admixture, but rather an intimate mixture usually formed by mixing the silicon dioxide with a suspension or slurry of microcrystalline cellulose and drying the mixture, such as by spray drying. The amount of silicon dioxide is normally within the range of 0.1 to 20 wt %, preferably from about 0.5 to 10 wt %, more typically from 1.25 to 5 wt %, and conveniently about 2 wt %, based on the weight of the silicified cellulose. The silicon dioxide generally has an average particle size not greater than 100 microns and typically between 5 and 50 microns. The microcrystalline cellulose is not particularly limited and generally has an average particle size in the range of 20 to 200 microns. Silicified cellulose is commercially available, for example, under the brand name PROSOLV from Penwest. For example, PROSOLV 50 and PROSOLV 90 are commercially available silicified (2% Si) microcrystalline celluloses having a median particle size of 50 and 90 microns, respectively, and are conveniently used in the present invention. Surprisingly, ProSolv 50 generally has an inferior taste/feeling in the mouth in comparison to ProSolv 90. Thus, silicified microcrystalline cellulose having a median particle size in the range of 75 to 125, especially about 90 microns, are likely preferred from this perspective.

The silicified cellulose serves as a matrix-forming excipient and is present in an amount of at least 50%, typically 50% to 90%, more typically 60% to 85%, of the total tablet mass. In some embodiments, the combined amount of simvastatin and silicified cellulose account for at least 80%, typically at least 85%, and usually in the range of 85-90% of the tablet mass.

While it is possible for the orally disintegratable tablets of the present invention to contain no lubricant, and hence avoid the sodium stearyl fumarate-induced instability, typically a lubricant is needed as a practical matter in the tabletting process. In these situations, the lubricant is generally a non-alkali lubricant. A “non-alkali lubricant,” as used herein, means that the lubricant does not contain an alkali metal or an alkaline earth metal in its molecule. Suitable non-alkali lubricants include glyceryl behenate, hydrogenated vegetable oil, talc, stearic acid, polyethylene glycol, poloxamer, mineral oil light, glyceryl palmitostearate, and glyceryl monostearate, but are not limited thereto. Commonly preferred prior art lubricants such as sodium stearyl fumarate and magnesium stearate are thus not within the scope of a non-alkali lubricant owing to the presence of a sodium and magnesium atom, respectively. The amount of the lubricant is generally within the range of 0.1 to 5% and typically about 0.5 to 2.0% based on the total weight of the tablet.

The orally disintegratable tablets of the present invention typically, though not necessarily, contain a disintegrant. The disintegration property of silicified cellulose may be enhanced by the presence of one or more disintegrants and/or superdisintegrants. Examples include an hydroxypropyl cellulose (HPC), especially low substituted hydroxypropyl cellulose (L-HPC) as defined in USP, alginic acid, microcrystalline cellulose, powdered cellulose, chitosan, colloidal silicon dioxide, guar gum, methylcellulose, pregelatinized starch, starch, sodium starch glycollate, carboxymethyl cellulose, and crosspovidone. For some embodiments, non-ionic disintegrants are preferred. Generally the amount of the disintegrant is within the range of 0.1 to 20%, more typically from 0.5% to 15%, still more typically 0.5% to 10% of the tablet mass. Disintegrants may be intragranular, extragranular, or both, as described in more detail hereinafter in the process of making the tablet.

Another useful excipient for a simvastatin orally disintegrating tablet is an antioxidant. Simvastatin is quite sensitive towards aerial oxidation. Typically, an antioxidant such as butylated hydroxyanisol (BHA) can be present in small amounts such as 0.01 to 2%.

The orally disintegratable tablets of the invention can have additional excipients including, among others, binders such as PVP, maize starch, or HPC; fillers; taste masking agents; natural or artificial sweeteners (e.g., aspartame, sucralose, etc.); flavors (e.g., mint flavor); colourants, etc. Typically, however, effervescent excipients are excluded from the composition. Similarly, water soluble binders are generally not included in the tablet composition.

The orally disintegratable tablets of the present invention can be made by any suitable tabletting technique. Because simvastatin presents certain handling problems in manufacturing tablets, it is often convenient to pre-granulate the simvastatin with a binder and optionally other intragranular excipients such as a disintegrant and/or antioxidant to form a granulate. The granulation is generally performed using wet granulation and usually using water as the liquid media. The simvastatin is preferably the micronized simvastatin as specified above.

The simvastatin-containing granules are mixed with the silicified microcrystalline cellulose, and any other excipients, usually an extra granular disintegrant, flavourant/colourant, and lubricant to form a tablet blend and then compressed into tablets. The process of making the tablet composition does not require the use of compounds or processes for improving the porosity or permeability of the tablet matrix. Thus, pore forming agents, foaming agents and the like are normally not used in making tablet compositions of the invention.

Besides the composition of the tablet, the disintegration time can be influenced by the size, shape, surface area, and hardness of the tablet. In general, tablets having larger surface areas and/or diameters have faster disintegration times. Conversely, tablets that have high hardness values generally have slow disintegration times. Accordingly, smaller tablets such as those containing 5-20 mg of simvastatin and having a total weight of 50 to 200 mg generally, though not necessarily, are formed with lower compression force to have a lower hardness, such as in the range of about 20 to 40 N, in order to achieve the desired disintegration time. Larger tablets such as those having40 to 80 mg of simvastatin and a total weight of 250 to 600 mg can have a higher hardness including up to 60 N or more, while still achieving the desired disintegration time. The shape of a tablet includes round, oval, and polygonal, e.g. pentagonal, octagonal, etc., which can be flat or biconvex. Additionally, the tablet may be scored and/or inscribed. Due to the presence of silicified cellulose, the friability of the tablet is generally less than 1.0%, such as less than 0.5%, or less than 0.2%, as measured according to Pharmacopeia Europea 2.9.7.

The rapidly disintegratable tablets of the invention provide a process for quickly releasing the simvastatin from a solid tablet. Specifically, in a preferred embodiment, the tablets can be used by placing them in a water environment for up to 30 seconds. In 30 seconds or less the tablet is disintegrated in the water environment, i.e. the tablet is no longer in existence or present in the water environment, albeit a residue thereof may be present. The water environment can be any moist environment including an oral cavity, a container of water such as the disintegration apparatus or a glass of water, etc. In case of a glass of water or other similar water container, a patient may consume the product after, or even during, disintegration. In this way, the once solid dosage form is consumed as essentially a liquid, including a suspension or slurry. When administering, one or more tablets may be used in order to achieve the intended dose of the active agent. Such multiple tablets can be given simultaneously or sequentially, normally within a few minutes of each other. In any event, the orally disintegratable tablets of the present invention can be used to treat high cholesterol in a mammal by directly orally administering the tablet to the patient or by pre-dissolving the tablet in a liquid such as water, juice, tea, or other beverage to form a simvastatin-containing liquid and then orally administering the liquid to the patient.

The disclosure in each of the above-mentioned patents and applications is incorporated herein by reference in its entirety. The present invention will be further illustrated by way of the following Examples. These Examples are non-limiting and do not restrict the scope of the invention.

### EXAMPLES

Granulates

Three granulates, G1-G3, were made having the compositions as shown in Table 1.

TABLE 1
Formulation of the granulate

| | G1 | G2 | G3 |
|---|---|---|---|
| Simvastatin | 90.8 | 90.8 | 90.8 |
| Butylated | 0.2 | 0.2 | 0.2 |
| hydroxyanisole | | | |
| Crospovidone | 3 | — | — |
| Sodium starch | — | 3 | 3 |
| glycolate | | | |
| Povidone | 6 | 6 | 6 |
| Water (ml) | 45 | 637.5 | 675 |

All values are in percent.
The G1 batch contains 120 grams of simvastatin.
The G2 and G3 batch contain 1800 grams of simvastatin.

All values are in percent. The GI batch contains 120 grams of simvastatin. The G2 and G3 batches contain 1800 grams of simvastatin.

Granulate GI was prepared in the Mi-Pro Procept using a 1900 ml bowl. Micronized simvastatin (d₉₀<5 micron), butylated hydroxyanisole and crospovidone were mixed for 2 minutes, (impellor speed 800 rpm, chopper 1000 rpm). The povidone (pvp) was dissolved in water and added in 2 minutes. The resulting blend was mixed until a granulate was obtained. The granulate was dried for 5 min with an inlet air temperature of 70° C. The product temperature was maximal 40° C. The granulate GI was milled and sieved through a 0.2 mm sieve using a Fritsh Pulveisrette 14.

Granulate G2 and G3 were made with a similar process but on a larger scale. Specifically, the Granulate G2 was prepared in the Diosna 25. Micronized simvastatin, butylated hydroxyanisole and sodium starch glycolate were put in the bowl of the Diosna and were mixed for 1 minute (impellor speed 170 rpm). The povidone (pvp) was dissolved in water and added in 1 minute. The blend was mixed until a granulate was obtained. The granulate was dried with an inlet air temperature of 70° C. until a product temperature of 40° C. was reached for G2 and a product temperature of 43° C. was reached for G3. Granulate G2 was milled through a Glatt conical high speed sieve (0.1 mm sieve) and continuously through a hammermill (0.1 mm sieve). Granulate G3 was milled through a Glatt conical high speed sieve (0.8 mm sieve).

Tablets

Using the granulates G1-G3 four tablet compositions were prepared as shown below:

TABLE 2
Orally Disintegrating Tablets

| | T1 | T2 | T3 | T4 |
|---|---|---|---|---|
| | (comparative) | (inventive) | (inventive) | (comparative) |
| Granulate G1 | 15.7 | 15.7 | — | — |
| (ppvp) | | | | |
| Granulate G2 | — | — | 15.7 | — |
| (ssg) | | | | |
| Granulate G3 | — | — | — | 15.7 |
| (ssg) | | | | |
| L-HPC | 6.0 | 6.0 | 6.0 | 6.0 |
| Silicified | 73.7 | 74.2 | 74.2 | 73.62 |
| cellulose | | | | |
| Sucralose | 0.5 | 0.5 | 0.5 | 0.5 |
| Mint | 2.5 | 2.5 | 2.5 | 2.5 |
| Iron oxide Red | 0.1 | 0.1 | 0.1 | 0.15 |
| Sodium stearyl | 1.5 | — | — | 1.5 |
| fumarate | | | | |
| glyceryl | — | 1 | 1 | — |
| behenate | | | | |
| (Compritol ™) | | | | |

All values are in percent.

The tablets were made as follows. For tablets T1-T3, the iron oxides were first mixed with 10% of the silicified cellulose and continuously de-agglomerated through a 180 micron sieve. The sucralose and mint were pre-blended and sieved through a 500 micron sieve. The milled granulate, L-HPC, silicified cellulose, the iron oxide and mint/sucralose preblend were mixed for 20 min in the turbula at 22 rpm. The lubricant was sieved through a 800 micron sieve, added to the blend, and the blend was mixed for another 5 min. The tablets were compressed into 70 mg tablets (10 mg of simvastatin) with a diameter of 7 mm and a hardness of 25 N.

For tablets T4, the iron oxides were first mixed with 10% of the silicified cellulose and continuously de-agglomerated through a 180 micron sieve. The mint was pre-blended with silicified cellulose and sieved through a 500 micron sieve. The milled granulate, L-HPC, silicified cellulose, the iron oxide pre blend, the sucralose and mint preblend were mixed for 30 min in Erweka kubus blender. The lubricant was sieved through a 800 micron sieve, added to the blend and the blend was mixed for another 5 min. The tablets were compressed into 140 mg tablets (20 mg of simvastatin) with a diameter of 9.5 mm and a hardness of 30 N.

Results

a) Disintegration

Table 3 shows the disintegration time of tablets T1-T4. The change in disintegrant and lubricant caused no significant disintegration difference.

TABLE 3
Disintegration time

| No. | Time (sec) |
|---|---|
| T1 | 5 |
| T2 | 3 |
| T3 | 3 |
| T4 | 9 |

b) Stability

The tablets T1-T3 and separately T4 were subjected to various stability tests at elevated temperature and relative humidity. The initial impurity levels, at time t=0, are shown in Table 4. Tables 5 and 6 show the impurity levels after one month of storage at the indicated conditions.

TABLE 4
Impurities at t = 0

| Impurity | T1 | T2 | T3 | T4 |
|---|---|---|---|---|
| Acid form of | 0.19 | 0.09 | 0.09 | 0.34 |
| simvastatin¹ | | | | |
| Epi-lovastatin | 0.30 | 0.30 | 0.21 | 0.21 |
| Dehydrated | <0.05 | <0.05 | 0.05 | 0.05 |
| simvastatin² | | | | |
| Dimer | 0.13 | 0.13 | 0.13 | 0.15 |
| Largest | 0.09 | 0.09 | 0.11 | 0.11 |
| unidentified | | | | |
| Total impurity | 0.52 | 0.42 | 0.38 | 0.65 |

Impurities in % Area/Area.
^{1,2}These compounds have the following structural formula, respectively:

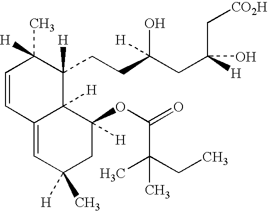

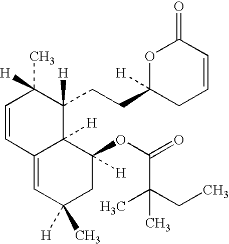

TABLE 5
Impurities at t = 1 month at 50° C./80% humidity

| | Open Dish | | | | HDPE with desiccant | | | |
|---|---|---|---|---|---|---|---|---|
| Impurity | T1 | T2 | T3 | T4 | T1 | T2 | T3 | T4 |
| Acid form of | 1.59 | 0.60 | 0.58 | 1.31 | 0.31 | 0.27 | 0.24 | 1.09 |
| simvastatin | | | | | | | | |
| Epi-lovastatin | 0.30 | 0.31 | 0.21 | 0.21 | 0.31 | 0.31 | 0.21 | 0.21 |
| Dehydrated | 0.42 | 0.13 | 0.17 | 0.36 | 0.13 | 0.09 | 0.07 | 0.47 |
| simvastatin | | | | | | | | |
| Dimer | 0.12 | 0.13 | 0.13 | 0.12 | 0.16 | 0.14 | 0.13 | 0.14 |
| Largest | 0.09 | 0.09 | 0.10 | 0.11 | 0.09 | 0.09 | 0.11 | 0.11 |
| unidentified | | | | | | | | |
| Total impurity | 2.42 | 1.07 | 1.09 | 2.01 | 0.80 | 0.70 | 0.65 | 1.97 |

Impurities in % A/A

TABLE 6
Impurities at t = 1 month at 40° C./75% humidity

| | Open Dish | | | | HDPE with desiccant | | | |
|---|---|---|---|---|---|---|---|---|
| Impurity | T1 | T2 | T3 | T4 | T1 | T2 | T3 | T4 |
| Acid form of | 1.52 | 0.95 | 0.91 | 0.93 | 0.23 | 0.18 | 0.15 | 0.64 |
| simvastatin | | | | | | | | |
| Epi-lovastatin | 0.32 | 0.30 | 0.21 | 0.20 | 0.31 | 0.31 | 0.21 | 0.21 |
| Dehydrated | 0.19 | 0.11 | 0.13 | 0.14 | 0.06 | 0.05 | 0.05 | 0.15 |
| simvastatin | | | | | | | | |
| Dimer | 0.14 | 0.17 | 0.16 | 0.14 | 0.14 | 0.13 | 0.13 | 0.15 |
| Largest | 0.09 | 0.09 | 0.10 | 0.11 | 0.09 | 0.09 | 0.10 | 0.11 |
| unidentified | | | | | | | | |
| Total impurity | 2.13 | 1.49 | 1.46 | 1.42 | 0.63 | 0.56 | 0.48 | 1.10 |

Impurities (in % A/A)

The inventive tablets T2 and T3, both using compritol as the lubricant instead of sodium stearyl fumarate, display less increase in (1) the acid form of simvastatin, (2) the dehydrated simvastatin and (3) total impurity level, in comparison to tablets T1 and T4. The presence of the sodium stearyl fumarate lubricant is the apparent cause of the instability. Note that other sodium-containing excipients, specifically the intra-granular disintegrant sodium starch glycollate, do not have an appreciable adverse effect on the stability: compare T2 with T3. This is true even though the sodium starch glycollate is more intimately associated with the simvastatin as an intra-granular disintegrant than the extra-granular lubricant. Thus, removing the lubricant sodium stearyl fumarate improves the stability of the orally disintegratable tablets. While the stability performance of T2 and T3 are comparable, T3 tablets are slightly preferred due to manufacturing preferences. Interestingly, other studies indicate that the comparison compositions perform even less well when stored in HDPE without a desiccant.

In view of the above description of the invention, it will be readily apparent to the worker skilled in the art that the same may be varied in many ways without departing from the spirit of the invention and such modifications are included within the scope of the present invention as set forth in the following claims.

We claim:

1. An orally disintegratable pharmaceutical tablet comprising an effective amount of simvastatin, at least 50 wt % of a silicified microcrystalline cellulose, and a non-alkali lubricant.

2. The tablet according to claim 1, wherein said non-alkali lubricant is selected from the group consisting of glyceryl behenate, hydrogenated vegetable oil, talc, stearic acid, polyethylene glycol, poloxamer, mineral oil light, glyceryl palmitostearate, and glyceryl monostearate.

3. The tablet according to claim 2, wherein said non-alkali lubricant is glyceryl behenate and said tablet further comprises BHA, L-HPC and crosspovidone.

4. The tablet according to claim 1, which further comprises a disintegrant, and/or an antioxidant.

5. The tablet according to claim 1, wherein said tablet does not contain sodium stearyl fumarate.

6. The tablet according to claim 1, wherein said effective amount of simvastatin is within the range of 2 to 200 mg.

7. The tablet according to claim 2, wherein said simvastatin is contained in an amount of 5 to 80 mg.

8. The tablet according to claim 1, wherein said simvastatin has a d₉₀≦5 microns.

9. The tablet according to claim 1, which further comprises L-HPC and crosspovidone and wherein said non-alkali lubricant is glyceryl behenate.

10. The tablet according to claim 1, which has a hardness of 20N to 60N.

11. The tablet according to claim 1, which has a friability of less than 1%.

12. The tablet according to claim 1, wherein said tablet does not contain a water soluble binder.

13. The tablet according to claim 1, which further comprises at least one additional excipient selected from the group consisting of taste masking agents, sweeteners, flavorants, and colourants.

14. A method of treating high cholesterol in a mammal, which comprises orally administering to a patient in need thereof a tablet according to claim 1.

15. A method of treating high cholesterol in a mammal, which comprises, disintegrating a tablet according to claim 1 in a liquid to a form a simvastatin-containing liquid; and orally administering said simvastatin-containing liquid to a patient in need thereof.

16. In an orally disintegratable tablet comprising simvastatin, a silicified microcrystalline cellulose, and a lubricant, the improvement of which comprises that said composition does not contain sodium stearyl fumarate.
